(19) United States
(12) Patent Application Publication
(10) Pub. No.: US 2006/0069361 A1
(43) Pub. Date: Mar. 30, 2006

(54) Absorbent article component having applied graphic, and process for making same

(75) Inventor: Christopher P. Olson, Neenah, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc.

(21) Appl. No.: 10/953,973

(22) Filed: Sep. 29, 2004

(51) Int. Cl. A61F 13/15

(52) U.S. Cl. 604/361

(57) ABSTRACT

In general, a process according to one embodiment thereof for manufacturing a substrate having a graphic applied thereto comprises applying a graphic to a substrate in a first configuration of the graphic. The substrate is then elongated in a direction such that the graphic is altered to a second configuration different from the first configuration. An absorbent article component according to one embodiment of the present invention comprises a substrate having apertures formed therein and being liquid permeable. A graphic is applied to the substrate at the apertures such that the apertures are disposed in at least a portion of the graphic.

### FIELD OF THE INVENTION

The present invention relates generally to absorbent articles intended for personal wear, and more particularly to absorbent articles having one or more components to which a graphic is applied.

### BACKGROUND OF THE INVENTION

Absorbent articles such as diapers, training pants, incontinence garments, and the like conventionally include a liquid permeable body-facing liner (also often referred to as a topsheet), an outer cover (also often referred to as a backsheet, an absorbent structure (also often referred to as an absorbent body or absorbent core), and in some instances, graphics visible from exterior of the article (e.g., on an exterior surface of the article). The graphics may provide a decorative feature, and particularly when used in connection with children's training pants, may be used to make the pants appear similar to conventional underwear. Further, the graphics may also be used to encourage training and/or be used to make the training experience a generally positive experience. For example, the graphics may be used to allow the caregiver to interact with the wearer in the training setting.

Accordingly, the graphics may take various forms, such as in the form of a character, object and/or alphanumeric (e.g., numbers, words, phrases, instructions, etc.), and the like. Moreover, at least some of the graphics may be “active graphics” configured to be capable of appearing or disappearing when the article is exposed to liquid, such as urine. These graphics can alert the wearer and the caregiver to the occurrence of urine in the article (i.e., an “accident”), and can assist in the training process. Reference may be made to U.S. Pat. No. 6,297,424, incorporated by reference herein for all purposes, for additional background information regarding graphics visible on the external surface of the article.

Nonetheless, such graphics, in certain circumstances, may not be completely satisfactory. For example, in some configurations, appearing or disappearing graphics visible on the exterior of the article may require a considerable amount of liquid, or multiple accidents, before the graphics are caused to appear or disappear. Additionally, graphics visible on the exterior of the article do not necessarily motivate the wearer to pull the article up and down for inspection, which can be a key training step.

To this end, absorbent articles having graphics visible from an interior surface of the article are disclosed in co-assigned U.S. patent application Ser. No. 10/881,255 entitled “ABSORBENT ARTICLE HAVING AN INTERIOR GRAPHIC AND PROCESS FOR MANUFACTURING SUCH ARTICLE”, filed Jun. 30, 2004, the entire disclosure of which is incorporated herein by reference. In particular, such interior graphics may be active graphics that change in appearance upon liquid insult within the absorbent article. For example, a fading graphic may be disposed on one or more components within the article, such as the absorbent structure, surge layer, liner, or a separate graphic layer that is disposed within the article. Upon liquid insult, the ink from the graphic dissolves and is taken into the absorbent structure along with the urine.

To allow the urine and ink mixture to flow to the absorbent structure, the component to which the graphic is applied may desirably be liquid permeable, so that as the ink dissolves in the urine the urine and ink mixture flows through the component, e.g., instead of back toward the liner where the ink may contact the wearer's skin. However, when printing on liquid permeable substrates, such as in flexographic printing process, there is a risk that upon application of ink to the substrate some of the ink will undesirably strike through the liquid permeable substrate and onto the printing drum of the printer.

### SUMMARY OF THE INVENTION

In general, a process according to one embodiment thereof for manufacturing a substrate having a graphic applied thereto comprises applying a graphic to a substrate in a first configuration of the graphic, the substrate is then elongated in a direction such that the graphic is altered to a second configuration different from the first configuration.

In another embodiment of the process, a substrate is delivered to a printing apparatus and the printing apparatus is operated to apply a graphic to the substrate, with the graphic having a length and a width. The substrate is then elongated to at least one of: increase the length of the graphic and increase the width of the graphic on the substrate.

An absorbent article component according to one embodiment of the present invention comprises a substrate having apertures formed therein and being liquid permeable. A graphic is applied to the substrate at the apertures such that the apertures are disposed in at least a portion of the graphic.

In general, an absorbent article according to one embodiment of the present invention comprises a liquid permeable topsheet, a backsheet, and an absorbent structure disposed between the topsheet and the backsheet. A substrate is disposed between the topsheet and the absorbent structure. The substrate has apertures formed therein, is liquid permeable, and has an active graphic applied thereto.

In another embodiment, an absorbent article has an inner surface arranged for facing a wearer of the article and an outer surface opposite the inner surface. The article generally comprises a backsheet at least in part defining the outer surface of the article, a topsheet in opposed relationship with the backsheet and at least in part defining the inner surface of the article, and a component disposed between the backsheet and the topsheet. The component comprises a substrate having a graphic thereon visible from the inner surface of the article. The substrate also has apertures therein and is liquid permeable.

Other features of the invention will be in part apparent and in part pointed out hereinafter.

### BRIEF DESCRIPTION OF THE DRAWINGS

FIG. 1 is a side perspective of one embodiment of an article of the present invention shown in the form of a pair of training pants having a mechanical fastening system fastened on one side of the training pants and unfastened on the opposite side thereof;

FIG. 2 is a bottom plan view of the training pants of FIG. 1 with the pants in an unfastened, unfolded and laid flat condition, and showing the surface of the training pants that faces away from the wearer;

FIG. 3 is a top plan view similar to FIG. 2 showing the inner surface of the training pants that faces the wearer when worn and with portions cut away to show underlying features;

FIG. 3A is a view similar to FIG. 3 but showing a second embodiment of the training pants;

FIG. 4 is a schematic section taken in the plane including line 4-4 of FIG. 3;

FIG. 5 is a schematic section similar to FIG. 4 but showing a third embodiment of the training pants;

FIG. 6 is a schematic section similar to FIG. 4 but showing a fourth embodiment of the training pants;

FIG. 7 is a schematic section similar to FIG. 4 but showing a fifth embodiment of the training pants;

FIG. 8 is a schematic section similar to FIG. 4 but showing a sixth embodiment of the training pants;

FIG. 9 is a schematic section similar to FIG. 4 but showing a seventh embodiment of the training pants;

FIG. 10 illustrates a side perspective of a seventh embodiment of the training pants;

FIG. 11 is a top plan view of the graphic layer of the absorbent article of FIG. 3 at an intermediate step of one embodiment of a process of the present invention for making an absorbent article component having a graphic thereon, with the graphic in a foreshortened configuration; and

FIG. 12 is a top plan view similar to FIG. 11 at a subsequent step of a process of the present invention for making an absorbent article wherein the substrate on which the graphic appears is stretched to elongate the foreshortened graphic of FIG. 11.

Corresponding reference characters indicate corresponding parts throughout the drawings.

### DETAILED DESCRIPTION

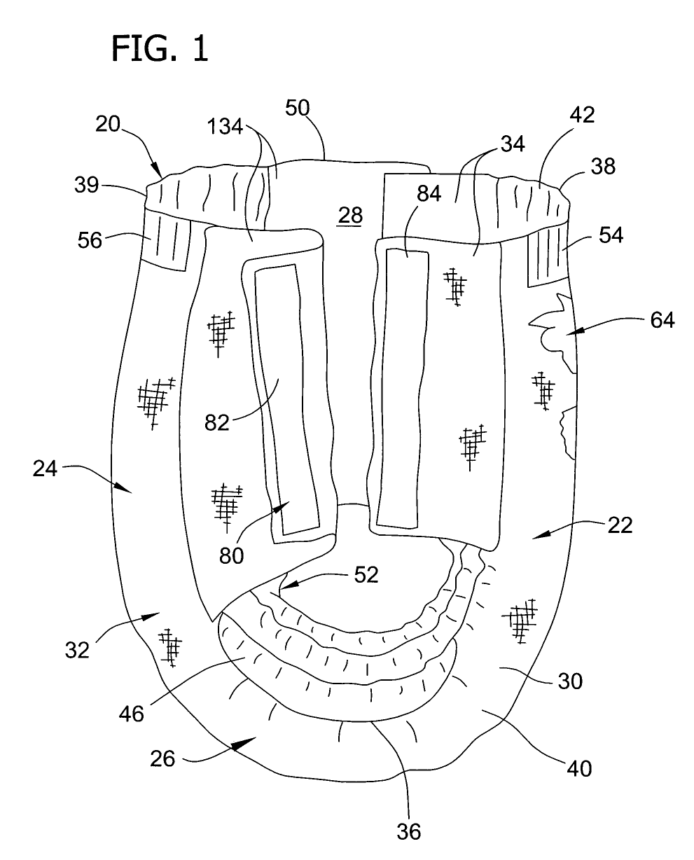

The present invention is generally directed to an absorbent article component having a graphic applied thereto. With reference generally to the drawings and in particular to FIG. 1, an absorbent article incorporating such a component of the present invention is representatively illustrated therein in the form of children's toilet training pants and is indicated in its entirety by the reference numeral 20. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are fully incorporated herein by reference.

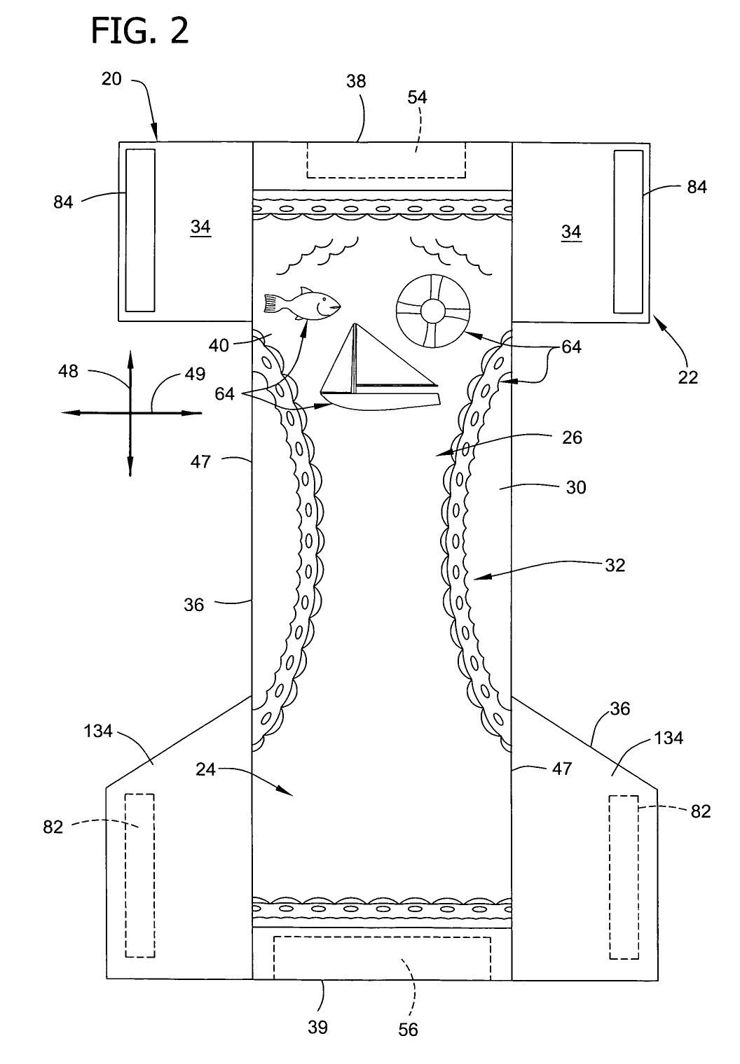

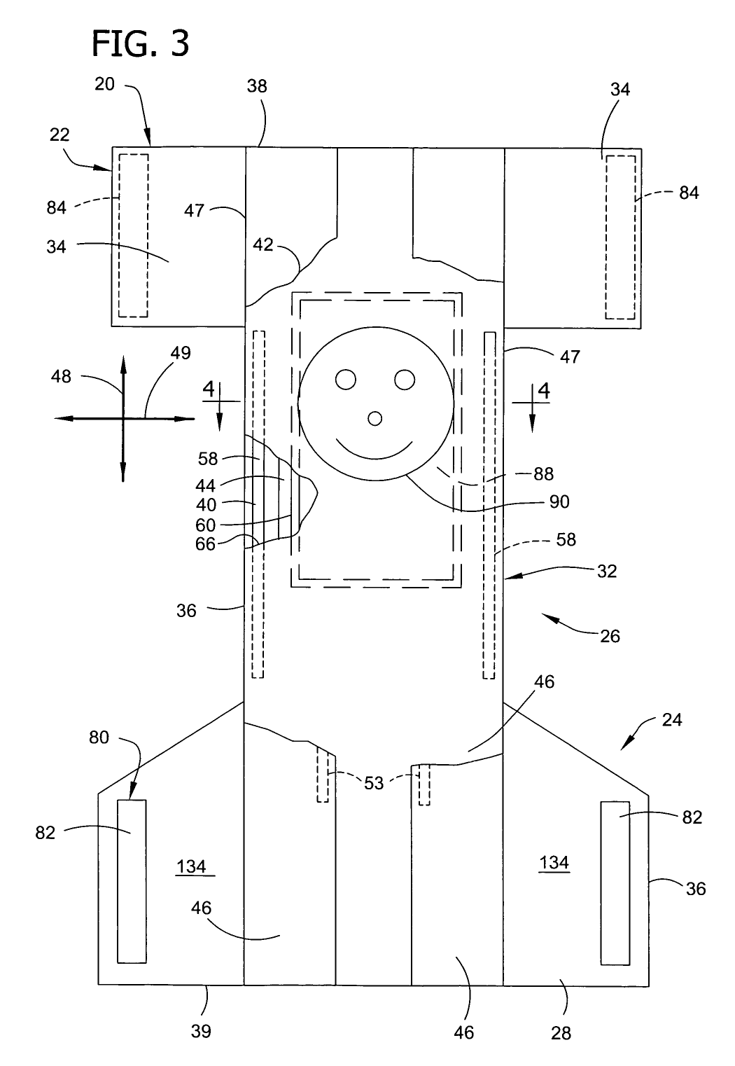

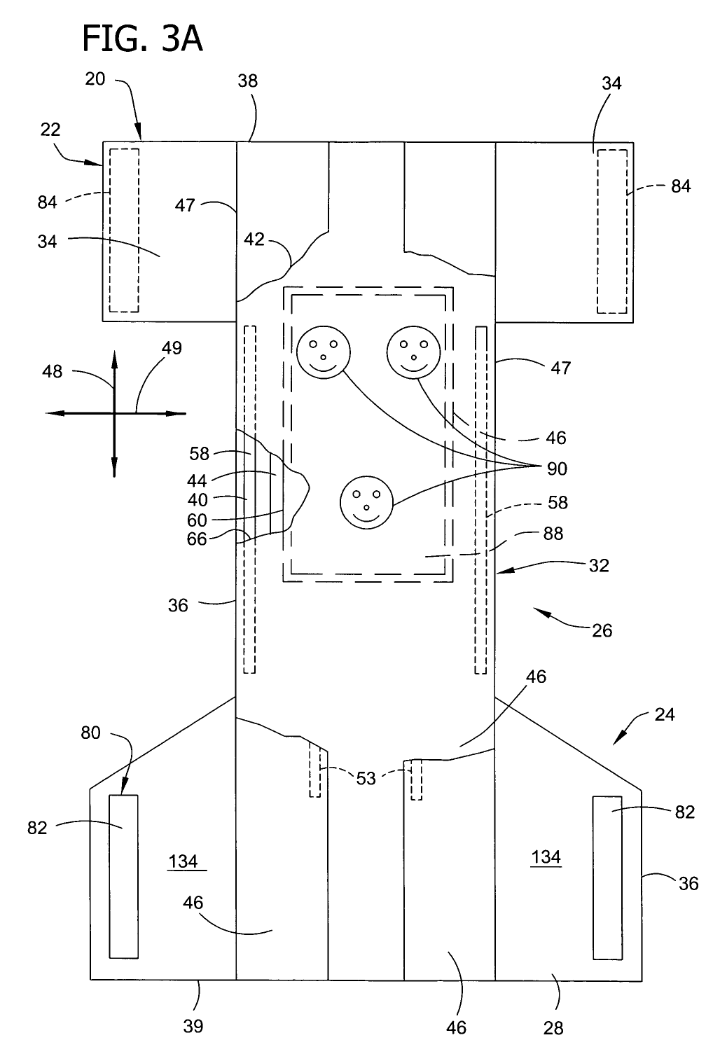

The pants 20 define a longitudinal direction 48 (FIG. 2) of the pants and a lateral direction 49 thereof perpendicular to the longitudinal direction as shown in FIGS. 2 and 3. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The pants 20 also define an inner surface 28 which during wearing of the pants faces the wearer, and an outer surface 30 opposite the inner surface and facing away from the wearer. The front and back waist regions 22, 24 comprise those portions of the pants 20 which, when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. With additional reference to FIGS. 2 and 3, the pair of training pants 20 has a pair of laterally opposite side edges 36 and a pair of longitudinally opposite waist edges (broadly, longitudinal ends), respectively designated front waist edge 38 and back waist edge 39.

The illustrated pants 20 comprises an absorbent assembly, generally indicated at 32, side panels 34, 134, and a fastening system, generally indicated at 80, for securing the pants in a three-dimensional pants configuration as partially illustrated in FIG. 1. In the embodiment of FIGS. 1-3, the training pants 20 comprises a generally rectangular central absorbent assembly 32 and the side panels 34, 134 are formed separately from and secured to the central absorbent assembly to extend laterally outward therefrom. For example, the side panels 34, 134 are permanently bonded to the central absorbent assembly 32 in the respective front and back waist regions 22 and 24 of the pants 20. More particularly, the front side panels 34 can be permanently bonded to and extend transversely outward beyond side margins 47 of the absorbent assembly 32 at the front waist region 22, and the back side panels 134 can be permanently bonded to and extend transversely outward beyond the side margins of the absorbent assembly at the back waist region 24. The side panels 34 and 134 may be bonded to the absorbent assembly 32 using attachment means known to those skilled in the art such as adhesive, thermal or ultrasonic bonding.

The front and back side panels 34 and 134, upon wearing of the pants 20, thus comprise the portions of the training pants 20 which are positioned on the hips of the wearer. The front and back side panels 34 and 134 can be permanently bonded together to form the three-dimensional configuration of the pants 20, or be releasably connected with one another such as by the fastening system 80 of the illustrated embodiment.

The side panels 34, 134 may suitably comprise an elastic material capable of stretching at least in a direction generally parallel to the lateral direction 49 of the training pants 20. Suitable elastic materials, as well as one process of incorporating elastic side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference. Alternatively, the side panel material may include other woven or non-woven materials, such as those described later herein as being suitable for construction of an outer cover 40 and/or a bodyside liner 42 of the pants; mechanically pre-strained composites; or stretchable but inelastic materials.

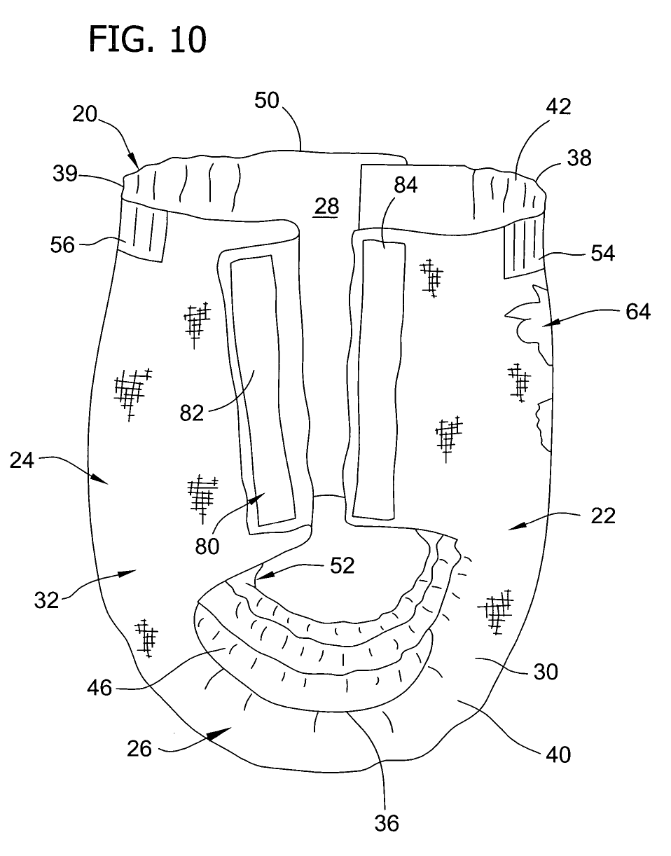

The absorbent assembly 32 is illustrated in FIGS. 1-3 as having a rectangular shape. However, it is contemplated that the absorbent assembly 32 may have other shapes (e.g., hourglass, T-shaped, I-shaped, and the like) without departing from the scope of this invention. Also, as shown in the embodiment of FIG. 10, the side panels 34, 134 may instead be formed integrally with the absorbent assembly, such as by being an extension of the outer cover 40, the bodyside liner 42, and or other components of the pants.

The absorbent assembly 32 comprises the outer cover 40 and the bodyside liner 42 (FIGS. 1 and 3) which is suitably attached to the outer cover 40 in a superposed relation therewith by adhesives, ultrasonic bonds, thermal bonds, pressure bonds, or other conventional techniques. The liner 42 of the illustrated embodiment in part defines the inner surface 28 of the article which faces the wearer of the article. The absorbent assembly 32 further comprises an absorbent structure 44 (FIG. 3) disposed between the outer cover 40 and the bodyside liner 42 for absorbing liquid body exudates exuded by the wearer, and a pair of containment flaps 46 secured to the bodyside liner 42 for inhibiting the lateral flow of body exudates.

With the training pants 20 in the fastened position as partially illustrated in FIG. 1, the front and back waist regions are connected together by the fastening system 80 to define the three-dimensional pants configuration having a waist opening 50 and a pair of leg openings 52. The front and back waist edges 38 and 39 (e.g. longitudinal ends) of the training pants 20 are configured to encircle the waist of the wearer to define the waist opening 50 (FIG. 1) of the pants.

As illustrated in FIG. 3, a flap elastic member 53 can be operatively joined with each containment flap 46 in any suitable manner as is well known in the art. The elasticized containment flaps 46 define a partially unattached edge which assumes an upright configuration in at least the crotch region 26 of the training pants 20 to form a seal against the wearer's body. The containment flaps 46 can be located along the side edges, and can extend longitudinally along the entire length of the absorbent assembly 32 or may extend only partially along the length thereof. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pants 20 may comprise a front waist elastic member 54, a rear waist elastic member 56, and leg elastic members 58, as are known to those skilled in the art (FIG. 2). The waist elastic members 54 and 56 may be operatively joined to the outer cover 40 and/or the bodyside liner 42 adjacent the longitudinal ends 38, 39. The leg elastic members 58 may be operatively joined to the outer cover 40 and/or the bodyside liner 42 along the opposite side edges generally at the crotch region 26 of the training pants 20.

The fastening system 80 of the illustrated embodiment comprises laterally opposite first fastening components 82 adapted for refastenable engagement to corresponding laterally opposite second fastening components 84. In one embodiment, a front or outer surface of each of the fastening components 82, 84 comprises a plurality of engaging elements. The engaging elements of the first fastening components 82 are adapted to repeatedly engage and disengage corresponding engaging elements of the second fastening components 84 to releasably secure the pants 20 in its three-dimensional configuration.

The fastening components 82, 84 can comprise any refastenable fasteners suitable for absorbent articles, such as adhesive fasteners, cohesive fasteners, mechanical fasteners, or the like. In particular embodiments the fastening components comprise mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like. Examples of suitable fastening systems are disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The outer cover 40 suitably comprises a material that is substantially liquid impermeable, such as a single layer of liquid impermeable material or more suitably a multi-layered laminate structure in which at least one of the layers is liquid impermeable. For instance, the outer cover 40 can comprise a liquid permeable outer layer and a liquid impermeable inner layer that are suitably joined together by a laminate adhesive, ultrasonic bonds, thermal bonds, pressure bonds or the like. Suitable laminate adhesives, which can be applied continuously or intermittently as beads, a spray, parallel swirls, or the like, can be obtained from Bostik Findley Adhesives, Inc., of Wauwautosa, Wis., U.S.A., or from National Starch and Chemical Company, Bridgewater, N.J. U.S.A. The liquid permeable outer layer can be any suitable material and is desirably one that provides a generally cloth-like texture. One example of such a material is a 20 gsm (grams per square meter) spunbond polyolefin nonwoven web. The outer layer may also be made of those materials of which the liquid permeable bodyside liner 42 is made. While it is not a necessity for the outer layer to be liquid permeable, it is suitable that it provides a relatively cloth-like texture to the wearer.

The inner layer of the outer cover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outer cover 40 when a single layer, prevents waste material from wetting articles, such as bed sheets and clothing, as well as the wearer and caregiver. A suitable liquid impermeable film for use as a liquid impermeable inner layer, or a single layer liquid impermeable outer cover 40, is a 0.75 mil (0.02 millimeter) polyethylene film commercially available from Pliant Corporation of Schaumburg, Ill., U.S.A.

Optionally, the outer cover 40 may be stretchable, and in some embodiments it may be elastomeric. As used herein, the term “stretchable” refers to a material that may be extensible or elastomeric. That is, the material may be extended, deformed or the like, without breaking, and may or may not significantly retract after removal of an extending force. The terms “elastomeric” or “elastic” are used interchangeably herein and refer to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a significant retractive force. In particular, elastomeric materials may be elongated/extended in at least one direction without breaking by at least 25% (to a length of at least 125%) of its initial unstretched length in at least one direction, and more suitably by at least 50% (to a length of at least 150%) of its initial unstretched length and which will recover, upon release of the applied stretching or biasing force, at least 10% of their elongation. It is generally preferable that the elastomeric material or composite be capable of being elongated by at least 100%, and more suitably by at least 200%, of its relaxed length and recover at least 30% and more suitably about 50% of its elongation upon release of a stretching, biasing force, within about one minute.

Similarly, extensible or elongatable materials of the present invention may be capable of elongating in at least one direction without breaking by at least 25% (to a length of at least 125% of its initial unstretched length) in at least one direction, and more suitably by at least 50% (to a length of at least 150% of its initial unstretched length), and even more suitably by at least 100% (to a length of at least 200% of its initial unstretched length). As an example, an extensible material having an initial unstretched length of 3 inches (7.6 centimeters) may be stretched without breaking to a stretched length of at least 3.75 inches (9.5 centimeters) in at least one direction (i.e., by at least 25%).

The outer cover 40 may be constructed of spunbond fabrics, films, meltblown fabrics, elastic netting, microporous web, bonded carded webs or foams provided by elastomeric or polymeric materials. Elastomeric non-woven laminate webs can comprise a non-woven material joined to one or more gatherable non-woven webs, films, or foams. Stretch Bonded Laminates (SBL) and Neck Bonded Laminates (NBL) are examples of elastomeric composites. Non-woven substrates (i.e., fabrics, webs, etc.) are fibrous substrates that are formed without the use of textile weaving processes that produce a structure of individual fibers interwoven in an identifiable repeating manner.

Examples of suitable outer cover materials are spunbond-meltblown fabrics, spunbond-meltblown-spunbond fabrics, spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from Invista, Inc. of Wilmington, Del.), KRATON elastomer (available from Kraton Polymers of Houston, Tex.), or strands of LYCRA elastomer (available from Invista, Inc. of Wilmington, Del.), or the like, as well as combinations thereof. The outer cover 40 may comprise materials that have elastomeric properties through a mechanical process, printing process, heating process, or chemical treatment. For example such materials may be apertured, creped, neck-stretched, heat activated, embossed, micro-strained, or combinations thereof and may be in the form of films, webs, and laminates.

Alternatively, the outer cover 40 may comprise a woven or non-woven fibrous web layer that has been totally or partially constructed or treated to impart the desired levels of liquid impermeability to selected regions that are adjacent or proximate the absorbent structure. For example, the outer cover 40 may include a gas-permeable, non-woven fabric layer laminated to a polymer film layer which may or may not be gas-permeable. Other examples of fibrous, cloth-like outer cover 40 materials can include a stretch thinned or stretch thermal laminate (STL) material composed of a 0.6 mil (0.015 mm) thick polypropylene blown film and a 0.7 osy (23.8 gsm) polypropylene spunbond material (2 denier fibers).

Suitable materials for a biaxially stretchable (i.e., stretchable both laterally and longitudinally) outer cover 40 include biaxially extensible material and biaxially elastic material. One example of a suitable biaxially stretchable outer cover material can include a 0.3 osy polypropylene spunbond that is necked 60% in the lateral direction 49 and creped 60% in the longitudinal direction 48, laminated with 3 grams per square meter (gsm) Bostik-Findley 2525A styrene-isoprene-styrene based adhesive to 8 gsm PEBAX 2533 film with 20% TiO₂ concentrate.

Another example of a suitable material for a biaxially stretchable outer cover 40 is a breathable elastic film/nonwoven laminate, described in U.S. Pat. No. 5,883,028, issued to Morman et al., incorporated herein by reference. Examples of materials having two-way stretchability and retractability are disclosed in U.S. Pat. No. 5,116,662 issued to Morman and U.S. Pat. No. 5,114,781 issued to Morman, both of which are hereby incorporated herein by reference. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The bodyside liner 42 is suitably compliant, soft-feeling, and non-irritating to the wearer's skin. The bodyside liner 42 is also sufficiently liquid permeable to permit liquid body exudates to readily penetrate through its thickness for flow to the absorbent structure 44. A suitable bodyside liner 42 may be manufactured from a wide selection of web materials, such as porous foams, reticulated foams, apertured plastic films, woven and non-woven webs, or a combination of any such materials. For example, the bodyside liner 42 may comprise a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The bodyside liner 42 may also be stretchable, and more suitably it may be elastomeric. Suitable elastomeric materials for construction of the bodyside liner 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon of Cleveland, Ohio), or PEBAX elastomers.

As an additional example, in one aspect the bodyside liner 42 suitably comprises a non-woven, spunbond polypropylene fabric composed of about 2 to 3 denier fibers formed into a web having a basis weight of about 12 gsm which is necked approximately 60 percent. Strands of about 9 gsm KRATON G2760 elastomer material placed eight strands per inch (2.54 cm) are adhered to the necked spunbond material. The fabric is surface treated with an operative amount of surfactant, such as about 0.6 percent AHCOVEL Base N62 surfactant, available from ICI Americas, a business having offices in Wilmington, Del., U.S.A. The surfactant can be applied by any conventional means, such as spraying, printing, brush coating or the like. Other suitable materials can be extensible biaxially stretchable materials, such as a neck stretched/creped spunbond. The bodyside liner 42 can also be made from extensible materials as are described in U.S. patent application Ser. No. 09/563,417 filed on May 3, 2000 by Roessler et al. or from biaxially stretchable materials as are described in U.S. patent application Ser. No. 09/698,512 filed on Oct. 27, 2000 by Vukos et al., both references which are herby incorporated by herein by reference.

The absorbent structure 44 is disposed between the outer cover 40 and the bodyside liner 42, which can be joined together by any suitable means such as adhesives, ultrasonic bonds, thermal bonds, or the like. While the illustrated absorbent structure 44 is shown and described herein as extending from the crotch region 26 into both the front and back waist regions 22 and 24, it is contemplated that the absorbent structure may extend from the crotch region into only the front waist region, or only the back waist region, without departing from the scope of this invention.

The absorbent structure 44 is suitably compressible, conformable, non-irritating to a wearer's skin, and capable of absorbing and retaining liquids and certain body wastes. For example, the absorbent structure 44 may comprise cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent structure comprises a matrix of cellulosic fluff and superabsorbent hydrogel-forming particles. The cellulosic fluff may include a blend of wood pulp fluff. One suitable type of fluff is identified with the trade designation CR 1654, available from U.S. Alliance of Childersburg, Ala., USA, and is a bleached, highly absorbent sulfate wood pulp containing primarily soft wood fibers.

The materials may be formed into a web structure by employing various conventional methods and techniques. For example, the absorbent structure 44 may be formed by a dry-forming technique, an air forming technique, a wet-forming technique, a foam-forming technique, or the like, as well as combinations thereof. Methods and apparatus for carrying out such techniques are well known in the art. Furthermore, the absorbent structure 44 may itself encompass multiple layers in a Z-direction (e.g., thickness) of the absorbent structure. Such multiple layers may take advantage of differences in absorbent capacity, such as by placing a lower absorbent capacity material layer and/or a surge-type material closer to the liner 42 and a higher absorbent capacity material closer to the outer cover 40. Likewise, discrete portions of a single-layered absorbent structure may encompass higher capacity absorbents, and other discrete portions of the structure may encompass lower capacity absorbents.

Superabsorbent material may be present in the absorbent structure 44 in an amount of from about 0 to about 90 weight percent based on total weight of the absorbent structure. The absorbent structure 44 may suitably have a density within the range of about 0.10 to about 0.35 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and preferably is capable of absorbing more than about 25 times its weight in liquid. Suitable superabsorbent materials are readily available from various suppliers.

The absorbent structure 44 may alternatively comprise a coform material. The term “coform material” generally refers to composite materials comprising a mixture or stabilized matrix of thermoplastic fibers and a second non-thermoplastic material. As an example, coform materials are made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene. Some examples of such coform materials are disclosed in U.S. Pat. Nos. 4,100,324 to Anderson, et al.; U.S. Pat. No. 5,284,703 to Everhart, et al.; and U.S. Pat. No. 5,350,624 to Georger, et al.; which are incorporated herein by reference.

The absorbent structure 44 may be stretchable so as not to inhibit the stretchability of other components to which the absorbent structure may be adhered, such as the outer cover 40 and bodyside liner 42. In a particularly suitable embodiment, the bodyside liner 42, the outer cover 40, and the absorbent structure 44 are each stretchable so that the absorbent structure allows for increased stretchability of the absorbent article as a whole. That is, non-stretchable absorbent structures tend to inhibit stretching of the outer cover and liner, even where the outer cover and liner are stretchable. A stretchable absorbent structure allows the outer cover and liner to more readily stretch, thereby increasing the overall stretchability (and ease of stretching) the entire article.

For this purpose, the absorbent structure material can include elastomeric fibers in an amount which is at least a minimum of about 2 wt %. The amount of elastomeric fibers can alternatively be at least about 3 wt %, and can optionally be at least about 5 wt % to provide improved performance. In addition, the amount of elastomeric fibers can be not more than about 60 wt %. Alternatively, the amount of elastomeric fibers can be not more than about 45 wt %, and optionally, can be not more than about 30 wt % to provide improved benefits. The elastomeric fiber content may impact the absorbent structure 44 stretchability and structural stability without excessively degrading the physical properties or the liquid-management properties of the absorbent structure. An absorbent structure 44 comprising an excessively low proportion of elastomeric fibers may be insufficiently stretchable, and one with an excessively high proportion of elastomeric fibers may exhibit an excessive degradation of its absorbent characteristics, such as poor intake, poor distribution and poor retention of liquid.

Examples of suitable stretchable absorbent structures are described in international patent application WO 03/051254 and U.S. Pat. Nos. 5,964,743, 5,645,542, 6,231,557, and 6,362,389 B1, each of which are incorporated by reference herein.

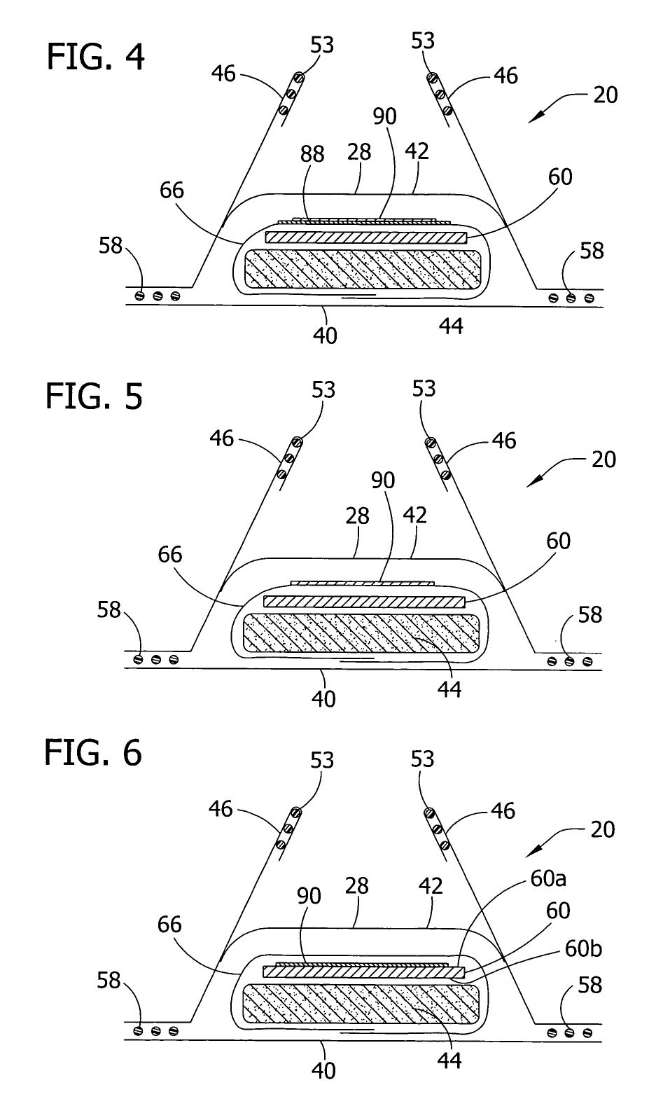

In some embodiments, such as that shown in FIG. 4, a surge management layer 60 may be disposed between the absorbent structure 44 and the liner 42, and may or may not be attached to various components of the article 20 such as the absorbent structure and/or the bodyside liner 42. The surge management layer 60 is intended to decelerate and diffuse surges or gushes of liquid that may be rapidly introduced into the absorbent structure 44 of the article 20. Desirably, the surge management layer 60 can rapidly accept and temporarily hold the liquid prior to releasing the liquid into the storage or retention portions of the absorbent structure 44. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein.

It is also contemplated that a surge layer material may be formed integrally with the absorbent structure 44, such as during initial air forming/air laying or other forming of the absorbent structure. For example, fibers deposited at the inner surface of the absorbent structure 44 may be different from those deposited throughout the remainder of the absorbent structure so that the inner surface defines an integrally formed surge layer.

Optionally, a substantially liquid permeable wrapsheet 66 may surround the absorbent structure, or both the absorbent structure and surge layer as shown in FIG. 4, to help maintain the integrity of the absorbent structure 44 upon wetting thereof. The wrapsheet 66 is typically placed about the absorbent structure 44 over at least the two major facing surfaces thereof. The wrapsheet 66 may comprise a polymeric non-woven such as spunbound, SMS, or the like or an absorbent cellulosic material, such as creped wadding, a high wet-strength tissue, or other non-woven material. The wrapsheet 66 can also be configured to provide a wicking layer that helps to rapidly distribute liquid to the absorbent fibers within the absorbent structure 44. The wrapsheet material 66 on one side of the absorbent structure 44 may be bonded to the wrapsheet located on the opposite side of the fibrous mass to effectively entrap the absorbent structure.

As shown in FIG. 3, the absorbent article 20 in accordance with one embodiment of the present invention comprises a liquid permeable graphic layer substrate 88 (broadly defining an absorbent article component) having a graphic 90 applied thereto. The graphic layer substrate 88 and graphic 90, which in the illustrated embodiment is in the form of a smiley face, are suitably positioned within the article 20 so as to be visible to the unaided human eye from the inner surface 28 of the article. The graphic layer substrate 88 is shown in FIGS. 3 and 4 as being disposed below the bodyside liner 42 of the article, e.g., between the liner and the absorbent structure 44, so that ink from the graphic 90 is not readily transferred to the skin of the wearer. In the various embodiments shown in the drawings, and in particular FIGS. 4-9, the thickness of the graphic 90 is exaggerated for illustrative purposes only, and does not reflect the actual or relative thickness of the graphic. It is contemplated that the graphic may form a layer on either surface of the substrate (i.e., either the body-facing surface or the absorbent-facing surface), or be incorporated or integrated within the substrate, and remain within the scope of this invention. Where the graphic 90 is positioned on the underside (i.e., absorbent-facing side) of the graphic layer substrate 88, the substrate may suitably be translucent or transparent.

In one particularly suitable embodiment, the graphic 90 comprises an active graphic and may suitably comprise one or more active graphics alone or in combination with one or more permanent graphics. As used herein, the term “active graphic” refers to a graphic that changes in appearance (to an unaided human eye) in response to liquid insult of the absorbent article. For example, the active graphic may be a fading graphic, which refers to a graphic that becomes invisible or significantly less visible when exposed to urine, or that becomes invisible or significantly less visible with the passage of time when exposed to a liquid insulted environment but not exposed directly to such an insult. In another embodiment, the active graphic may be an appearing graphic, which refers to a graphic that becomes visible or becomes significantly more visible when exposed to urine, or that becomes visible or becomes significantly more visible with the passage of time when exposed to a liquid insulted environment but not exposed directly to such an insult. In other embodiments, the active graphic may change colors when exposed to urine, or exposed to a liquid insulted environment but not exposed directly to such an insult.

Fading graphics may be suitably formed from an ink that is soluble in aqueous solutions such as urine. As such, the ink can be located in the article 20 so that it becomes wet and dissolves when the product is insulted with liquid. Suitable urine-soluble inks are available from a variety of commercial vendors, such as Sun Chemical Corp. of Philadelphia, Pa., USA under the trade designation AQUA DESTRUCT. Particular urine-soluble compositions are disclosed in U.S. Pat. No. 4,022,211 issued May 10, 1977 to Timmons et al., which is incorporated herein by reference to the extent it is consistent (i.e., not in conflict) herewith. The ink color can be selected to provide a pleasing appearance and graphic impact, including fading rapidly upon contact with liquid. In particular aspects, and to facilitate rapid fading, the fading graphics can comprise line drawings having a line width of from about 1 to about 5 millimeters.

Alternatively, the active graphic can be formed from a composition such as an ink or adhesive that changes color when exposed to an aqueous solution such as urine. A color change composition can be adapted to blend in with a background or surrounding color, either before or after exposure to the aqueous solution. Suitable compositions of this color-change type are available from a variety of commercial vendors, such as a pH-change/color-change hot melt adhesive available from Bostik-Findley Adhesives, Inc. of Wauwatosa, Wis., USA. Alternatively, the active graphic can comprise pH sensitive inks, fugitive inks, colored absorbent particles, hydratable salts, moisture sensitive films, enzymes, heat sensitive inks and dyes, or the like.

In one embodiment, the graphic 90 may suitably be activated (e.g., appear, fade or otherwise change color) in about 3 minutes or less, more suitably in about 1 minute or less, and still more suitably in about 20 seconds or less, when the absorbent article 20 is insulted with 200 milliliters or more of urine, more suitably when the absorbent article is insulted with about 40 to about 60 milliliters or more of urine, and even more suitably when the absorbent article is insulted with about 10 milliliters or more of urine.

In contrast to active graphics, the term “permanent graphic” is used herein to refer to a graphic that does not substantially change its degree of visibility when the absorbent article is insulted with urine in simulated use conditions. The change in visibility of a graphic or a portion of a graphic can be determined based on a person's observation of the graphic before and after the article containing the graphic is exposed to liquid. For purposes hereof, an article is exposed to liquid by immersing the article completely in an aqueous solution containing 0.9 weight percent sodium chloride, used at room temperature (≅23° C.), for a period of twenty minutes. After 20 minutes the product is removed from the aqueous solution and placed on a TEFLON™ coated fiberglass screen having 0.25 inch (6.35 mm) openings, which is commercially available from Taconic Plastics Inc., Petersberg, N.Y., USA, which in turn is placed on a vacuum box and covered with a flexible rubber dam material. A vacuum of 3.5 kilopascals (0.5 pounds per square inch) is drawn in the vacuum box for a period of 5 minutes, after which the article is removed and observed. The person with normal or corrected vision of 20-20 should make the observations from a distance of 1 meter in an environment providing 30 footcandles (320 Lux) of illumination. Changes in the visibility of the graphic should be identified, and distinguished where necessary from changes in the color of other materials such as fluff pulp within an absorbent assembly. Desirably, the permanent graphic can be configured so that the entire graphic also does not substantially change its appearance, size or shape when the product is insulted with liquid or exposed to the environment.

The graphic 90 on the graphic layer 88 may include, but is not limited to, scenes, characters, animals, objects, alphanumerics such as numbers, letters, words, phrases and the like. In particular aspects, the graphic 90 may also be gender specific; that is, the graphic may be generally considered to be of interest to boys or to girls.

The graphic 90 may be suitably applied to the graphic layer substrate 88 using a variety of printing methods. As an example, in a particularly suitable embodiment the graphic 90 may be imprinted on the graphic layer substrate 88 using a flexographic printing process. Flexographic printing is a conventional printing technique which uses flexible, raised rubber or photopolymer plates to carry an inked image to a substrate, such as the graphic layer 88, outer cover 40, liner 42, wrapsheet 66, surge layer 60, or absorbent structure 44. As an example, flexographic printing apparatus are shown and/or described in U.S. Pat. Nos. 5,458,590 (Schleinz et al.); U.S. Pat. No. 5,566,616 (Schleinz et al.); U.S. 2003/0019374A1 (Harte); and U.S. Pat. No. 4,896,600 (Rogge et al.). Further, the graphic 90 may be printed, sprayed, or otherwise applied to the graphic layer 88 by other suitable printing techniques (e.g., ink jet, rotogravure, etc.).

The graphic layer substrate 88 is suitably constructed to be liquid permeable for incorporation into the absorbent article. As used herein in reference to the graphic layer substrate 88 or other absorbent article component to which graphic 90 is applied in accordance with the present invention, the term liquid permeable means that the substrate is penetrative by fluid through the entire Z-directional (i.e., orthogonal to both the longitudinal and transverse directions 48, 49) thickness of the substrate with the substrate under pressure of about 10 millibars as determined by INDA (Association of the Nonwoven Fabrics Industry) IST 80.4 Standard Test Method for Water Resistance Using the Hydrostatic Pressure Test.

The graphic layer substrate 88 may be initially constructed to be liquid permeable or it may be generally liquid impermeable upon initial making thereof and rendered liquid permeable in subsequent processing. For example, in accordance with one suitable embodiment, the graphic layer substrate 88 or other substrate to which the graphic 90 is applied may comprise an otherwise liquid impermeable substrate having apertures formed therein in sufficient size and/or density to render the substrate liquid permeable.

The graphic layer substrate 88 is also suitably capable of being elongated in at least one direction, and may be capable of bi-directional elongation. As used herein, the term elongation means that the substrate is capable of increasing in size in the direction of elongation from a first configuration to a second, longer (or wider) configuration. In one suitable embodiment, the substrate 88 may be stretchable in at least one direction, and may be capable of bi-directional stretch. In another suitable embodiment, the substrate may be extensible in at least one direction, and may be bi-directionally extensible. For example, the graphic layer substrate 88 (or other substrate to which the graphic 90 is applied) may be a stretchable film, such as a polymeric film, microporous (e.g. breathable) film, etc. One suitable film is a polyethylene film available from Pliant Corp. of Chippewa Falls, Wis., U.S.A. having a basis weight of approximately 14 osy (ounces per square yard) and a thickness of about 0.75 mils. Other suitable films include, but are not limited to, PMP-1 film material available from Mitsui Toatsu Chemicals, Inc. of Tokyo, Japan and XKO-8044 polyolefin film available from 3M Company of Minneapolis, Minn., U.S.A.

In alternative embodiments, the graphic layer substrate 88 may be non-stretchable, e.g., inextensible, and remain within the scope of this invention. For example, the substrate 88 may be relatively inextensible and rendered capable of elongation by forming weakness elements in the substrate as described later herein.

In another suitable embodiment, the graphic layer substrate 88 may be a non-woven substrate. The non-woven substrate may be a single non-woven web or a laminate of non-woven webs, and may or may not be stretchable. Suitable non-woven substrates include spunbonded materials and spun-bonded/melt-blown/spun-bonded (SMS) laminates consisting of non-woven outer layers of spun-bonded polypropylene and an interior barrier layer of melt-blown polypropylene such as described in U.S. Pat. No. 5,213,881 issued to Timmons et al. The term spun-bonded fibers refers to small diameter fibers which are formed by extruding molten thermoplastic material as filaments from a plurality of fine capillaries of a spinnerette having a circular or other configuration, with the diameter of the extruded filaments then being reduced as described in, for example, U.S. Pat. No. 4,430,563 to Appel et al., U.S. Pat. No. 3,692,618 to Dorschner et al, U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Petersen, and U.S. Pat. No. 3,542,615 to Dobo et al. Spun-bonded fibers are generally continuous and may have average diameters larger than 7 microns, often between about 10 and 30 microns.

Other examples of suitable non-woven substrates include a stretch thinned or stretch thermal laminate (STL) material comprising of a 0.6 mil (0.015 mm) thick polypropylene blown film and a 0.7 osy (23.8 gsm) polypropylene spunbond material (2 denier fibers). Elastomeric materials, such as PEBAX elastomer (available from AtoChem located in Philadelphia, Pa.), HYTREL elastomeric polyester (available from Invista, Inc. of Wilmington, Del.), KRATON elastomer (available from Kraton Polymers of Houston, Tex.), or LYCRA elastomer (available from Invista, Inc. of Wilmington, Del.), or the like, as well as combinations thereof, could also be used. Biaxially stretchable (i.e., stretchable both laterally and longitudinally) materials may also be used. One example of a suitable biaxially stretchable material can include a 0.3 osy polypropylene spunbond that is necked 60% in the lateral direction 49 and creped 60% in the longitudinal direction 48, laminated with 3 grams per square meter (gsm) Bostik-Findley 2525A styrene-isoprene-styrene based adhesive to 8 gsm PEBAX 2533 film with 20% TiO₂ concentrate. It is also contemplated that the substrate 88 may comprise a laminate of a non-woven web and a film without departing from the scope of this invention. The non-woven substrate may also comprise a laminate of a non-woven layer laminated to a film, foam or another non-woven layer.

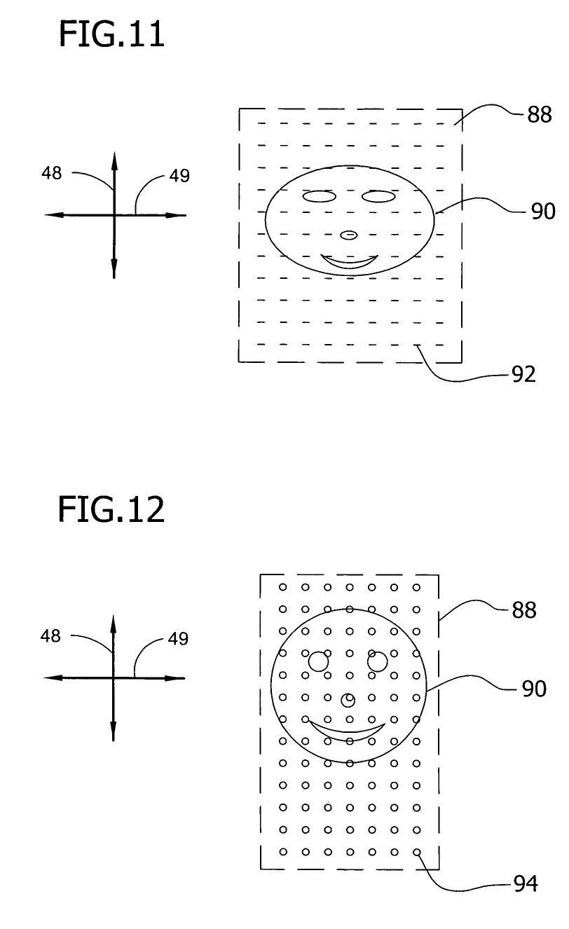

In accordance with one embodiment of the present invention for making a liquid permeable absorbent article component having a graphic, the graphic 90 is applied to the substrate 88 while the substrate is substantially liquid impermeable. In a flexographic printing process, printing the graphic 90 onto the liquid impermeable substrate 88 reduces the risk of undesirable strikethrough of the ink through the substrate and onto the printing drum. With reference to FIG. 11, in a particularly suitable embodiment the graphic 90 is applied to the substrate 88 in a foreshortened configuration of the graphic. The term foreshortened as used in reference to the graphic 90 means that the graphic is shorter in at least one dimension, e.g., the length and/or the width thereof, than an intended final dimension (e.g., as it ultimately appears when the component is incorporated into the article, i.e., as the article wearer and/or caregiver are intended to view the graphic). In such an embodiment, the graphic 90 may be foreshortened in both its length and width dimensions (i.e., lateral direction 49), or it may be foreshortened in one dimension and be otherwise applied in its intended final dimension, or it may be foreshortened in one dimension and lengthened (e.g. overdimensioned) in the other dimension. For example, in the illustrated embodiment of FIG. 11, the graphic 90 is foreshortened along its length dimension which extend in the longitudinal direction 48, i.e., it is shorter than it will appear as used in the absorbent article. The width of the graphic 90 shown in FIG. 11, which extends in the longitudinal direction 48, is greater than the width of the graphic as it will finally appear in the absorbent article as shown in FIG. 12.

Following printing of the graphic 90 onto the substrate 88, at least one and more suitably a number of weakening elements 92 are formed in the film. The term weakening elements as used herein means a reduced basis weight, thickness and/or density of the substrate, or an imperfection formed in the substrate, that substantially reduces the resistance to elongation and rupturing of the substrate at the weakness element. For example, the weakening elements 92 in the illustrated embodiment of FIG. 11 comprise perforations (and more particularly slits) formed through the thickness of the substrate 88. It is understood, however, that the perforations 92 may extend only partially through the thickness of the substrate without departing from the scope of this invention. The slits shown in FIG. 11 suitably extend in a direction transverse to the direction in which the substrate is to be elongated (e.g., transverse to the direction of foreshortening of the graphic, such as in the longitudinal direction of the substrate shown in FIG. 11) as discussed later herein. It is also contemplated that the perforations 92 may instead comprise small holes, of generally any shape, extending fully or partially through the thickness of the substrate, and remain within the scope of this invention.

The substrate 88 is then elongated in the direction or directions of foreshortening of the graphic 90 to elongate the graphic to its intended (e.g., unforeshortened) final dimensions. For example, the substrate may suitably be mechanically elongated, such as by suitable drawing or elongating apparatus. In the illustrated embodiment of FIG. 12, the substrate 88 has been neck-stretched in the longitudinal direction thereof to lengthen the initially foreshortened graphic 90. Neck-stretching the substrate 88 reduces the width of the substrate as it elongated, thereby decreasing the width of the graphic 90 (which had been overdimensioned in width upon application of the graphic to the substrate) to its intended final width. In embodiments where the substrate 88 is capable of bi-directional elongation, the graphic 90 may be foreshortened in both length and width and the substrate subsequently elongated both longitudinally and laterally to increase the length and width of the graphic to its intended final dimensions.

Upon elongating the substrate 88, apertures 94 form in the substrate at the locations of the weakening elements 92 to render the substrate liquid permeable. For example, where perforations 92 (e.g., slits) or other openings are formed through the thickness of the substrate 88 as shown in FIG. 11, the perforations or openings expand upon elongating of the substrate to form apertures 94 as shown in FIG. 12 sufficiently sized to permit liquid to pass therethrough to thereby render the substrate liquid permeable. Where the weakening elements 92 extend only partially through the substrate 88 or comprise other imperfections that weaken the substrate, elongating the substrate ruptures the substrate at the weakening elements to form apertures 94 in the substrate that further widen upon additional stretching of the substrate.

The apertures 94 in the substrate 88 are sufficiently sized, shaped and in sufficient number to allow liquids (e.g., urine) to flow through the substrate while not significantly diminishing the integrity of the graphic 90 printed thereon. As an example of the size of the apertures, each aperture may suitably have a cross-sectional area (e.g., across the opening as measured at the body-side facing surface of the substrate) in the range of from about 0.2 mm² to about 12.6 mm², more suitably about 0.8 mm² to about 7.1 mm², and even more suitably in the range of about 0.8 mm² to about 4.9 mm². The apertures may each suitably have a cross-sectional dimension (e.g., a diameter where the aperture is circular) measured at the body-side facing surface of the substrate in the range of about 0.5 mm to about 4 mm, more suitably in the range of about 1 mm to about 3 mm, and even more suitably in the range of about 1 mm to about 2.5 mm. The number of apertures may suitably be in the range of about 6 apertures per square inch to about 25 apertures per square inch.

An area ratio of the apertures at the graphic may be determined as the ratio of the total surface area of the bodyfacing surface of the substrate at the graphic applied thereto to the total area of all apertures formed in the substrate 88 at the graphic 90. In other embodiments, an area ratio of the apertures throughout the substrate may be determined as the ratio of the total surface of the body-facing surface of the substrate to the total area of all apertures formed in the substrate. It is understood that the substrate may be liquid permeable only over the area of the substrate to which the graphic 90 is applied, e.g., such that the area ratio of the apertures at the graphic may be different from the area ration the apertures at non-graphic regions of the substrate, and remain within the scope of this invention. As an example, the area ratio of the apertures over the substrate (or at least over the graphic portion of the substrate) is in the range of about 20:1 to about 5:1.

In another embodiment of a process for making the absorbent article component, the weakening elements 92 may formed in the substrate 88 prior to printing the graphic on the substrate instead of subsequent thereto. It is also contemplated that apertures 94 may instead be formed in the substrate 88 after elongating the substrate to elongate the foreshortened graphic. It is further contemplated that in other embodiments the substrate may not be apertured at all and may instead remain liquid impermeable upon elongating thereof to elongate the foreshortened graphic without departing from the scope of this invention.

In accordance with another embodiment of a process for making an absorbent article component, the foreshortened graphic 90 may be applied to a liquid permeable substrate 88. For example, the substrate may be a liquid permeable non-woven substrate. Weakening elements 92 are formed in the substrate 88 to extend partially or fully through the thickness thereof. Upon elongating the substrate 88 to elongate the foreshortened graphic 90, apertures 94 form in the substrate in a manner similar to that described above to increase the liquid permeability of the substrate relative to the permeability of substrate prior to elongation.

In yet another embodiment, the substrate 88 may be a non-woven substrate that is otherwise non-stretchable and may or may not be liquid permeable upon application of the foreshortened graphic thereto. No weakening elements 92 are formed in the non-stretchable substrate 88. Rather, pulling on the non-stretchable substrate 88 to elongate the foreshortened graphic 90 causes the fibers of the substrate to separate or tear apart from each other (e.g., similar to what happens when one pulls apart a cotton ball) to thereby render the substrate liquid permeable or to increase the liquid permeability of the substrate where the substrate was liquid permeable prior to elongation.

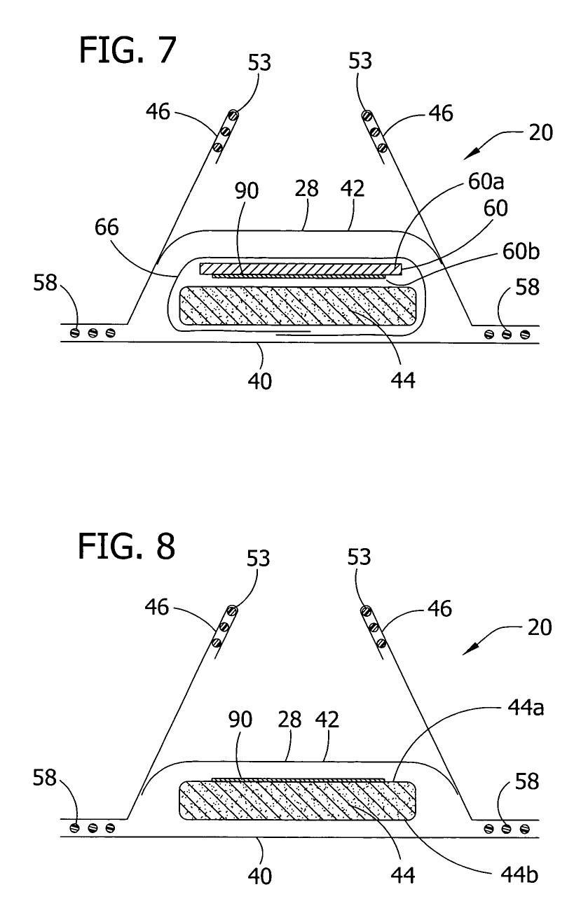

While the graphic 90 is described above as being applied to a graphic layer substrate 88 for incorporation into the absorbent article (e.g., pants 20), it is understood that the absorbent article component with applied graphic may comprise other components of the absorbent article. For example, in the embodiment of FIG. 5, the substrate to which the graphic 90 is applied is the wrapsheet 66, and more suitably the outer cover facing side of the wrapsheet so that it is free from direct contact with the bodyside liner 42 to thereby reduce the risk of ink being transferred to the skin of the wearer. FIG. 6 shows another embodiment in which the substrate to which the graphic 90 is applied is the surge layer 60, and more particularly a liner-facing surface 60a of the surge layer 60. In the embodiment of FIG. 6, the wrapsheet 66 wraps both the surge layer 60 and absorbent structure 44 to separate graphic 90 on the surge layer from the inner surface 28 of the article 20. In the alternative embodiment of FIG. 7, the graphic 90 is instead applied to a cover-facing surface 60b of the surge layer 60. The graphic 90 is separated from the inner surface 28 of the article 20 by the surge layer 60, the wrapsheet 66 and the bodyside liner 42. It is also contemplated that in either of the embodiments of FIGS. 6 and 7 the wrapsheet 66 may be omitted without departing from the scope of this invention.

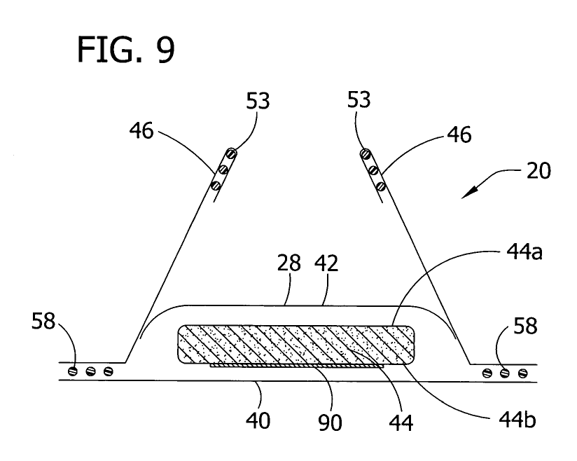

FIG. 8 illustrates an alternative embodiment in which the substrate to which the graphic 90 is applied is the absorbent structure 44, and more particularly a liner-facing surface 44a of the absorbent structure. In the alternative embodiment of FIG. 9, the graphic is instead applied to a cover-facing surface 44b of the absorbent structure 44. The absorbent structure 44 shown in FIG. 9 suitably has a central portion of reduced thickness at the location of the applied graphic 90 so that the absorbent structure is more translucent to thereby facilitate visibility of the graphic 90 from the inner surface 28 of the article 20. Alternatively, the materials from which the absorbent structure 44 is constructed may be modified by adding or omitting one or more additives (e.g., omitting a titanium dioxide additive) to render the absorbent structure more translucent and thereby increase the visibility of the graphic 90.

In order to provide a proper focus on the graphic 90, the graphic may be particularly positioned within the absorbent article 20. For example, in embodiments where the graphic 90 comprises an active graphic, the graphic may be positioned in a gender specific target zone for urination within the product to increase the likelihood that the graphic 90 will be activated upon insult of the article. As such, in the illustrated embodiment of FIGS. 3 and 3a, at least a portion of the graphic 90 is spaced from the front waist edge 38 in the longitudinal direction 48 by about 25% to about 50% of the article length. In another aspect, at least a portion of the graphic 90 may be spaced from the front waist edge 38 in the longitudinal direction 48 by about 35% to about 60% of the article length.

Moreover, each graphic 90 may define a total graphic area. In the illustrated embodiment, the total graphic area is equal to the area of the circular smiley face graphic 90 that may be calculated by squaring one-half of the diameter of the circle and multiplying by the constant pi (3.14). In other embodiments of the article 20, the graphic 90 may be generally rectangular or square and the total graphic area may be calculated by multiplying the largest dimension of the graphic in the longitudinal direction 48 by the largest dimension of the graphic 90 in the lateral direction 49. Suitably, the total graphic area may be at least 25 square cm, and more suitably at least 45 square cm. Alternatively, as illustrated in FIG. 3A, the pants 20 may include a plurality of graphics 90. Thus, the plurality of graphics 90 may, in total, define a total graphic area. The total graphic area may be calculated by adding the graphic area of each graphic 90. The plurality of graphics 90 may define a total graphic area of at least 25 square cm. Such total graphic areas as described above suitably draws the attention of the wearer and can therefore act as a more meaningful training aid.

As shown in FIGS. 1 and 2, the training pants 20 may also include one or more exterior graphics 64 disposed on the exterior article surface 30, such as by being applied to the outer cover 40. These exterior graphics 64 may include, but are not limited to, scenes, characters, animals, objects, alphanumerics such as numbers, letters, words, phrases and the like, highlighting or emphasizing leg and waist openings 52, 50 in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product.

The exterior graphics 64 are suitably formed on or applied to the outer cover 40 or another substrate bonded to or placed with or placed near the outer cover 40 by any suitable technique. For example, in one embodiment the exterior graphic 64 may be applied to the outer cover (or a layer thereof where the outer cover is constructed from multiple layers) in a foreshortened configuration and the outer cover then stretched in accordance with the process described previously herein. The exterior graphics 64 may also be registered with other components of the absorbent article 20 during manufacture such that the exterior graphics are positioned in the desired regions of the product.

It is contemplated that the exterior graphics 64 may be active graphics, permanent graphics, or combinations thereof. In particular aspects, at least one of the exterior graphics 64 is an active graphic, and more particularly a fading graphic. Exterior graphics 64 suitable for use with the present invention are described in U.S. Pat. No. 6,297,424 issued Oct. 2, 2001 to Olson, et al. and U.S. Pat. No. 6,307,119 issued Oct. 23, 2001 to Cammarota et al., the disclosures of which are incorporated herein to the extent they are consistent (i.e., not in conflict) herewith.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A process for manufacturing a substrate having a graphic applied thereto, said process comprising:
applying a graphic to a substrate in a first configuration of the graphic; and
elongating said substrate in a direction such that the graphic is altered to a second configuration different from said first configuration.

2. The process set forth in claim 1 wherein the first configuration of the graphic comprises a foreshortened configuration of said graphic, said elongating step comprising elongating said substrate in a direction that elongates the graphic to said second configuration of the graphic.

3. The process set forth in claim 1 wherein the second configuration comprises a predetermined final configuration of the graphic on the substrate.

4. The process set forth in claim 1 wherein the step of applying a graphic to the substrate comprises applying an active graphic to said substrate.

5. The process set forth in claim 4 wherein the applying step comprises applying a fading graphic to said substrate.

6. The process set forth in claim 1 wherein the step of applying a graphic to the substrate comprises applying a graphic to the substrate in a first configuration of the substrate, the elongating step comprising elongating said substrate to a second configuration longer than said first configuration, the substrate being liquid permeable in at least the second configuration thereof.

7. The process set forth in claim 6 wherein the substrate comprises a film, said process further comprising forming weakening elements in the film prior to the step of elongating the film, the elongating step comprising elongating the film to said second configuration thereof, said elongating of the film causing apertures to form in said film to thereby render the film liquid permeable.

8. The process set forth in claim 7 wherein the step of forming weakening elements in the film is performed after applying the graphic to the film.

9. The process set forth in claim 7 wherein the weakening elements extend through the full thickness of the film.

10. The process set forth in claim 6 wherein the substrate comprises a non-woven substrate, the non-woven substrate being elongatable from said first configuration of the substrate to said second configuration thereof.

11. The process set forth in claim 6 wherein the non-woven substrate has a first liquid permeability in the first configuration of the substrate and a second liquid permeability greater than said first liquid permeability in the second configuration of the substrate following elongation thereof.

12. The process set forth in claim 11 further comprising the step of forming weakening elements in the non-woven substrate before the step of elongating said substrate, the liquid permeability of the non-woven substrate increasing at least at said weakening elements upon elongating the substrate from the first configuration to the second configuration thereof.

13. The process set forth in claim 1 further comprising the step of forming at least one weakening element in the substrate prior to elongating said substrate, the elongating step comprising elongating the substrate to form at least one aperture in said substrate at said at least one weakening element, said at least one aperture being sized sufficient to permit liquid to flow through the substrate at said at least one aperture.

14. The process set forth in claim 13 wherein said step of forming at least one weakness element in said substrate comprises embossing said substrate.

15. The process set forth in claim 13 wherein said step of forming at least one weakness element in said substrate comprises forming at least one perforation in said substrate.

16. A process for manufacturing a substrate having a graphic thereon, said process comprising:
delivering a substrate to a printing apparatus;
operating the printing apparatus to apply a graphic to the substrate, said graphic having a length and a width; and
elongating said substrate to at least one of:
increase the length of the graphic and increase the width of the graphic on the substrate.

17. The process set forth in claim 16 wherein the step of delivering the substrate to the printing apparatus comprises delivering the substrate to a flexographic printing apparatus.

18. The process set forth in claim 16 wherein the elongating step comprises neck-stretching said substrate to increase the length of the graphic and decrease the width of the graphic on the substrate.

19. The process set forth in claim 16 wherein the step of applying the graphic to the substrate comprises applying an active graphic to said substrate.

20. The process set forth in claim 19 wherein the applying step comprises applying a fading graphic to said substrate.

21. The process set forth in claim 16 wherein the substrate is liquid permeable following said elongating step.

22. The process set forth in claim 21 wherein the substrate comprises a film, said process further comprising forming weakening elements in the film prior to the step of elongating the film, the elongating step comprising elongating the film to said second configuration thereof, said elongating of the film causing apertures to form in said film to thereby render the film liquid permeable.

23. The process set forth in claim 22 wherein the step of forming weakening elements in the film is performed after applying the graphic to the film.

24. The process set forth in claim 22 wherein the weakening elements extend through the full thickness of the film.

25. The process set forth in claim 21 wherein the substrate comprises a non-woven substrate.

26. The process set forth in claim 25 wherein the non-woven substrate has a first liquid permeability in a first configuration of the substrate and a second liquid permeability greater than said first liquid permeability in an elongated, second configuration of the substrate following elongation thereof.

27. The process set forth in claim 25 further comprising the step of forming weakening elements in the non-woven substrate before the step of elongating said substrate, the liquid permeability of the non-woven substrate increasing at least at said weakening elements upon elongating the substrate from the first configuration to the second configuration thereof.

28. The process set forth in claim 16 further comprising the step of forming at least one weakening element in the substrate prior to elongating said substrate, the elongating step comprising elongating the substrate to form at least one aperture in said substrate at said at least one weakening element, said at least one aperture being sized sufficient to permit liquid to flow through the substrate at said at least one aperture.

29. The process set forth in claim 28 wherein said step of forming at least one weakness element in said substrate comprises embossing said substrate.

30. The process set forth in claim 28 wherein said step of forming at least one weakness element in said substrate comprises forming at least one perforation in said substrate.

31. An absorbent article component, said component comprising a substrate having apertures formed therein and being liquid permeable, and a graphic applied to the substrate at the apertures such that the apertures are disposed in at least a portion of the graphic.

32. The absorbent article component set forth in claim 31 wherein the graphic applied to the substrate is an active graphic.

33. The absorbent article component set forth in claim 32 wherein the active graphic is a fading graphic.

34. The absorbent article component set forth in claim 32 wherein the active graphic is an appearing graphic.

35. The absorbent article component set forth in claim 31 wherein the substrate comprises a stretched substrate.

36. The absorbent article component set forth in claim 35 wherein the substrate comprises a stretched, liquid impermeable film having said apertures formed therein to render said substrate liquid permeable.

37. The absorbent article component set forth in claim 35 wherein the substrate comprises a stretched non-woven substrate.

38. The absorbent article component set forth in claim 37 wherein the substrate comprises a neck-stretched non-woven substrate.

39. The absorbent article component set forth in claim 31 wherein the apertures are sized in the range of about 0.5 mm to about 4 mm.

40. The absorbent article component set forth in claim 39 wherein the apertures are each sized in the range of about 1 mm to about 2.5 mm.

41. The absorbent article component set forth in claim 31 wherein the apertures have a total aperture area at the graphic and the substrate has a total surface area at the graphic, the ratio of the total surface area of the substrate at the graphic to the total aperture area at the graphic being in the range of about 20:1 to about 5:1.

42. An absorbent article comprising:
a liquid permeable topsheet;
a backsheet;
an absorbent structure disposed between the topsheet and the backsheet; and
a substrate disposed between the topsheet and the absorbent structure, said substrate having apertures formed therein and being liquid permeable, said substrate having an active graphic applied thereto.

43. The absorbent article set forth in claim 42 wherein the active graphic is a fading graphic.

44. The absorbent article set forth in claim 42 wherein the active graphic is an appearing graphic.

45. The absorbent article set forth in claim 42 wherein the substrate comprises a stretched substrate.

46. The absorbent article set forth in claim 42 wherein the substrate comprises a stretched, liquid impermeable film having said apertures formed therein to render said substrate liquid permeable.

47. The absorbent article set forth in claim 46 wherein the substrate comprises a stretched non-woven substrate.

48. The absorbent article set forth in claim 47 wherein the substrate comprises a neck-stretched non-woven substrate.

49. An absorbent article having an inner surface arranged for facing a wearer of the article and an outer surface opposite said inner surface, said article comprising:
a backsheet at least in part defining the outer surface of the article;
a topsheet in opposed relationship with the backsheet and at least in part defining the inner surface of the article; and
a component disposed between the backsheet and the topsheet, said component comprising a substrate having a graphic thereon visible from the inner surface of the article, said substrate having apertures therein and being liquid permeable.

50. The absorbent article set forth in claim 49 wherein said graphic is an active graphic.

51. The absorbent article set forth in claim 50 wherein said active graphic is a fading graphic.

52. The absorbent article of claim 50 wherein said active graphic is an appearing graphic.

53. The absorbent article of claim 49 wherein the component is selected from the group comprising an absorbent structure, a wrapsheet, a surge layer, a flow control layer and a graphic layer.

54. The absorbent article set forth in claim 49 wherein the substrate comprises a stretched substrate.

55. The absorbent article set forth in claim 54 wherein the substrate comprises a stretched, liquid impermeable film having said apertures formed therein to render said substrate liquid permeable.

56. The absorbent article set forth in claim 54 wherein the substrate comprises a stretched non-woven substrate.

57. The absorbent article set forth in claim 54 wherein the substrate comprises a neck-stretched non-woven substrate.
